(19) United States
(12) Patent Application Publication
(10) Pub. No.: US 2010/0121207 A1
(43) Pub. Date: May 13, 2010

(54) APPARATUS AND METHOD FOR DETECTING AN APNEA USING SIGNALS SENSED IN DEPENDENCE ON THE BLOOD PRESSURE

(75) Inventors: Hans-Joachim Moersdorf, Fuerth (DE); Stefan Aschenbrenner, Eckental (DE); Christian Hofmann, Erlangen (DE)

Correspondence Address: SCHOPPE, ZIMMERMANN , STOCKELER & ZINKLER;C/O KEATING & BENNETT, LLP, 1800 Alexander Bell Drive, SUITE 200, Reston, VA, 20191, US

(73) Assignee: Fraunhofer-Gesellschaft zur Foerderung der angewandten Forschung e.V., Munich (DE)

(21) Appl. No.: 12/597,533

(22) PCT Filed: Apr. 22, 2008
(86) PCT No.: PCT/EP08/03232

§ 371 Date: Dec. 8, 2009

(30) Foreign Application Priority Data
Apr. 27, 2007 (DE) 10 2007 020 038.4

(51) Int. Cl. A61B 5/02

(52) U.S. Cl. 600/500

(57) ABSTRACT

The existence of an apnea may be inferred when, in a pulse wave signal which is provided and is detected in dependence on the blood pressure, a change in a waveform of the pulse wave signal meets a predetermined detection criterion.

### BACKGROUND OF THE INVENTION

The present invention concerns the detection of sleep disorders, and particularly how an apnea can be detected by means of signals detected in dependence on the blood pressure.

Sleep disorders are a phenomenon occurring more and more frequently, heavily restricting the quality of life and capability of the peopled affected. Special types of occurring sleep disorders may further have a lasting detrimental effect on the patient's health.

Two sleep disorders occurring especially frequently are apneas and hypopneas. In case of an apnea, complete respiratory arrests occur, the frequency of which may vary within wide boundaries, with values of more than 200-300 of such sleep disorders per night not being rare. The occurrence of at least 10 respiratory arrests, each lasting for at least 10 seconds, within one hour of sleep is regarded as the general definition for the disease of apnea. Apnea may have several causes, with the most frequent one being occlusion of the upper respiratory tracts occurring during sleep (obstructive sleep apnea). The occlusion normally is caused by the windpipe collapsing or by the windpipe being closed off by the tongue. The velum, which also is responsible for snoring, among other things, may also be involved. If the velum relaxes, it may lead to the fact that it completely closes the respiratory tracts, so that the supply of oxygen to the lungs, and hence also to the brain, is interrupted. Due to the above connection, apnea often also is observed in people prone to heavy snoring.

The drop in the oxygen content of the blood triggers an alarm signal or counter measure in the brain after a certain time, so that the people concerned experience a short-term central nervous activation, a so-called arousal (comparable to a panic reaction), at the end of an apnea. In case of an arousal, the patient concerned typically is startled with a loud snoring noise, whereupon breathing starts again. The oxygen content may normalize. As described above, since this process repeats several times per night, it becomes obvious that sleep apnea may cause a series of negative side effects, such as increased fatigue during the daytime, reduced mental and physical capability, lack of concentration, headache, depressions, and the like.

Apart from obstructive apnea, so-called central sleep apnea often also is observed, wherein no occlusion of the respiratory tracts takes place, but which rather is due to a cessation of the breathing impulses on the part of the brain. Here, the observable course of the apnea until the arousal substantially is the same as of the obstructive apnea.

In summary, the clinical picture of the disease of sleep apnea may be characterized by recurrent respiratory arrests occurring during sleep. Sleep apneas, or apneas, may be roughly categorized into two groups, as has already been mentioned: central apneas and obstructive apneas. Central apneas involve defects, in the brain, in activating the breathing muscles, and in obstructive apneas, blockages in the pharyngeal and throat areas lead to an occlusion of the respiratory tracts. Apneas are usually detected in that several vital parameters of a patient to be examined are recorded during sleep, for example the breathing effort, the respiratory flow, or the oxygen saturation of the blood. An apnea may also be detected by means of an EEG. Since it is only with difficulty that an apnea can be detected, several of the above-described vital parameters are typically observed in combination so as to reach an unambiguous conclusion. Several methods may detect an apnea also when only a subset of the above vital parameters or a relatively small number of vital parameters are recorded.

Due to the large number of parameters to be evaluated, or to the complexity of the evaluation, apneas normally cannot be detected until after they have stopped, i.e. after an arousal has ended the apnea. The reasons for this can be found, on the one hand, in the basic difficulty of recognizing the beginning of an apnea in the vital parameters recorded, and/or in the high signal processing complexity.

### SUMMARY

According to an embodiment, an apparatus for detecting apneas may have: a provider for providing a blood pressure-dependent pulse wave signal; and an evaluator for evaluating the pulse wave signal provided so as to infer the existence of an apnea when a low-frequency portion of the pulse wave signal falls below a predetermined threshold value, the evaluator being configured to determine the time-variable threshold value for an evaluation time by averaging the low-frequency portion of the pulse wave signal within a time interval which comes before the evaluation time.

According to another embodiment, a method of detecting apneas may have the steps of: providing a blood pressure-dependent pulse wave signal; and evaluating the pulse wave signal provided so as to infer the existence of an apnea when a low-frequency portion of the pulse wave signal falls below a predetermined threshold value, the time-variable threshold value for an evaluation time being determined by averaging the low-frequency portion of the pulse wave signal within a time interval which comes before the evaluation time.

According to another embodiment, a computer program may have a program code for performing the method of detecting apneas, said method having the steps of: providing a blood pressure-dependent pulse wave signal; and evaluating the pulse wave signal provided so as to infer the existence of an apnea when a low-frequency portion of the pulse wave signal falls below a predetermined threshold value, the time-variable threshold value for an evaluation time being determined by averaging the low-frequency portion of the pulse wave signal within a time interval which comes before the evaluation time, when the program runs on a computer.

In accordance with an embodiment of the present invention, a photoplethysmographically detected pulse wave signal is evaluated so as to infer the existence of an apnea if a change in a low-frequency portion of the pulse wave signal meets a suitable detection criterion.

Evaluating photoplethysmographically detected pulse waves enables detecting an apnea at clearly reduced cost and in a more efficient and faster manner. As will become clear from the more detailed discussion of the embodiments of the invention in the paragraphs which follow, it is even possible, in this manner, to detect the beginning of an apnea. Some embodiments of the present invention make use of this in order to perform an alarm action which may involve, for example, stimulating a patient in whom the beginning of an apnea has been detected. By means of the timely stimulus, one may achieve that the apnea is ended prematurely before the body starts to experience a major oxygen debt.

In accordance with one embodiment of the present invention, a CPAP (continuous positive airway pressure) apparatus is activated, as an alarm measure, or the pressure at which said apparatus is operating is increased. CPAP devices are devices used for treating apneas, and they involve the patient wearing a mask connected to the CPAP device, which ensures that the patient is continuously ventilated and/or that the respiratory air supplied to the patient continuously exhibits a specified air pressure. This may prevent the windpipe from collapsing. Conventional CPAP devices have a number of unpleasant side effects, even though they may clearly reduce the frequency of apneas, in principle.

On the one hand, the windpipe is dehydrated by the continuous operation of the CPAP device, on the other hand, the patients frequently suffer from conjunctivitis, since it is not possible to apply the mask that may be used for ventilation to the patient in an absolutely tight-fitting manner. Thus, due to the overpressure generated within the CPAP device, air constantly escapes at the edges of the respiratory mask, which occurs, among other things, also in the direction of the eyes, which may lead to the above-described conjunctivitis. In one embodiment of the invention, in the case of apnea having been detected, the CPAP device is activated when it may really be useful. The CPAP device may not be switched on until this moment, or, alternatively, a CPAP device operating at low pressure (i.e. also having minor side effects) may be controlled such that the pressure is increased for a short time. By means of a system consisting of a CPAP device and an apparatus for detecting apneas, the side effects that occur when CPAP devices are employed may therefore be clearly reduced or avoided.

In a further embodiment, the low-frequency portion of the photoplethysmographically detected pulse wave signal which is to be analyzed may be generated by means of low-pass filtering using a cutoff frequency that is lower than the average pulse frequency.

In further embodiments of the present invention, a simple threshold-value comparison is used for detecting an apnea, or rather the beginning of an apnea, so that overall, signal processing of only low complexity may be used.

In a further embodiment of the invention, the cutoff frequency of the low-pass filter may be adaptively adjusted to the pulse, or to the pulse frequency, of the patient observed, so that it will be ensured at any time that the signal to be evaluated cannot be impaired by the typical temporal variation of the pulse frequency.

In a further embodiment of the present invention, a measure of the severity or acuteness of an apnea may be derived in a simple manner in that said severity or acuteness is determined in dependence on the difference from the currently valid threshold value.

Similarly, in a further embodiment, an apnea that has occurred can be differentiated from a hypopnea. If the value measured falls far short of the currently valid threshold value, this will indicate the existence of an apnea, whereas the case where said currently valid threshold value is slightly fallen short of may indicate the existence of a hypopnea. Consequently, an optionally connected CPAP device may remain switched off, for example, in the case of a hypopnea, since with a hypopnea it takes longer for the patient to enter into critical oxygen debt than with an apnea.

In a further embodiment, a simplified photoplethysmographic sensor is employed to record the photoplethysmographic pulse waves, said sensor operating only with light of a single wavelength range. Typical photoplethysmographic sensors use two wavelength ranges in parallel, so that in this embodiment, the material cost, or the cost of the hardware used, may be further reduced.

Other features, elements, steps, characteristics and advantages of the present invention will become more apparent from the following detailed description of preferred embodiments of the present invention with reference to the attached drawings.

### BRIEF DESCRIPTION OF THE DRAWINGS

Embodiments of the present invention will be explained below with reference to the accompanying figures, wherein:

FIG. 1 shows a photoplethysmographically captured pulse wave signal;

FIG. 2 shows a schematic analysis of a low-frequency signal portion;

FIG. 3 shows an example of an inventive apparatus for detecting apneas;

FIG. 4 shows a further example of an apparatus for detecting apneas; and

FIG. 5 shows an example of a method of detecting apneas.

### DETAILED DESCRIPTION OF THE INVENTION

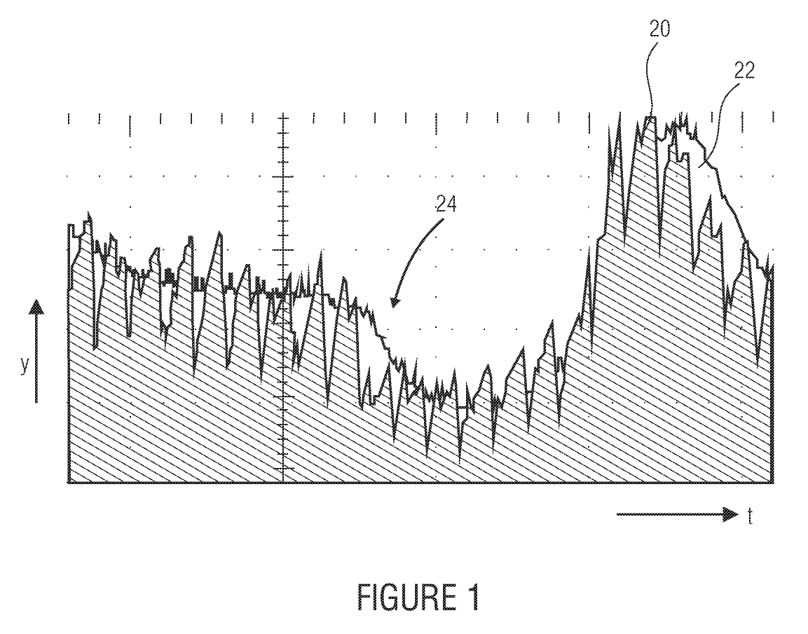

With reference to FIG. 1, photoplethysmographic detection of measurement data will be briefly set forth below, and on the basis thereof, the ideas based on several implementations of the invention will be developed.

FIG. 1 shows, on the x-axis, the time t in random units, and on the y-axis, a light intensity captured by means of a photoplethysmographic sensor, also in random units.

Photoplethysmographic detection of pulse waves is frequently used for non-invasive measurement of the oxygen content of the blood (SpO₂). To this end, a part of the body—frequently a finger, but sometimes other parts of the body such as earlobe, wrist or sternum—is irradiated by means of a modulated light source. Normally, light having two different wavelengths is used for determining the oxygen saturation of the blood.

In other fields of application, more than two different wavelengths may also be used, in some specific simple cases of application, one single wavelength may be employed.

Therefore, in photoplethysmography, a part of the body is generally irradiated with light, the intensity of the amount of light that can be received after the part of the body has been irradiated being determined as a measured quantity.

In the jagged curve 20, FIG. 1 shows the waveform, depicted by means of an oscillograph, of a photoplethysmographically detected pulse wave signal, i.e. essentially a detected light intensity of a transmitted-light photography of a part of a body. It is to be noted in this context that the light curve of a second light source that is usually employed is not shown in FIG. 1 for reasons of depictability. The smooth curve 22 shows a low-frequency portion of a pulse wave signal as may be generated, for example, by low-pass filtering the jagged signal 20.

The jagged curve 20 thus represents the curve of the current brightness, as it were, as may be formed by combining a DC voltage portion 22 and a dynamic portion. The individual spikes of the jagged curve 20 correspond to the individual pulse beats of the patient, which in the present example were observed in a finger of the patient. This specific curve shape results from the fact that with each pulse beat, the blood pressure in the person's artery system increases for a short time, whereby the diameters of the arteries increase slightly.

These variations of the diameters result in that the light being transmitted through the blood vessels moved travel a longer optical path length through the blood, which leads to increased absorption, so that each pulse beat leads to a short-term drop in intensity of the photoplethysmographically detected light or in the detected intensity. The smooth curve 22 is formed by temporally averaging the dynamic signal, i.e. the jagged curve 20, and it thus shows the DC voltage portion (DC component) of said signal. For some embodiments of the invention, it is sufficient to employ a simplified version of an SpO₂ plethysmograph, since only a single measured curve for light of a single wavelength needs to be evaluated in order to reliably infer apnea.

In photoplethysmographic measurement, the DC voltage portion or the low-frequency signal portion 22 changes over time due to the change of some parameters influencing the measurement, such as changing ambient light, a change in the position of the measurement location in relation to the blood vessel, the height of the measurement location above the heart, and changing sleeping positions of the patient to be examined. In addition, said portion 22 exhibits intense scattering from one individual to another, i.e. highly depends on the respective patient.

The inventors have found that in the case of an obstruction as may be caused, for example, by breathing with a closed nose and mouth, a spontaneous increase in the blood pressure may be observed as a reaction to the intrathoracic pressure variations. Since the underlying mechanisms of breathing disturbances are similar, the above also applies to central apneas.

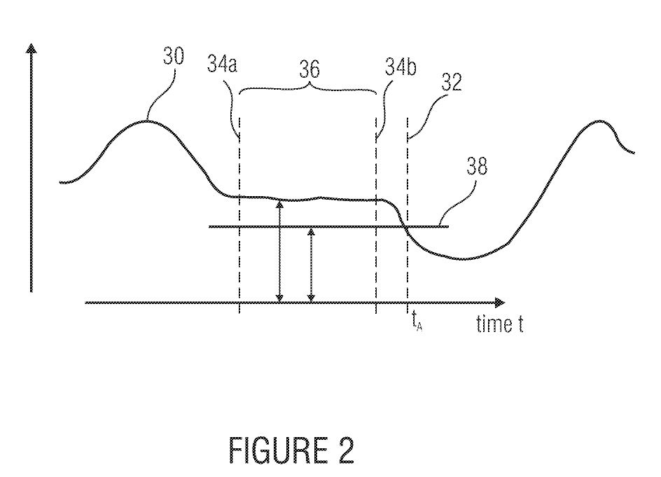

When the breathing ceases for a time as a consequence of an apnea, a spontaneous increase in the blood pressure therefore occurs, which takes place on a time scale which clearly differs from the time scale underlying the pulse beats. In the event of an occurrence of such changes in the blood pressure caused by apneas, said changes in the blood pressure will also result in an expansion of the vessels, which means that the cross section that the light beams need to pass increases. As a result, the DC voltage portion in the brightness curve is shifted, as is depicted in FIG. 1 at the time of the beginning of an apnea 24 at which the low-frequency portion of the pulse wave signal clearly drops. The drop in the low-frequency portion of the pulse wave signal, which occurs at the time 24, therefore enables immediate inference of the existence of an apnea. In this context, one may take into account that any changes in the DC voltage portion that may be observed may also be caused by the interference effects already mentioned. However, these two changes may be differentiated by extremely simple signal processing methods, for example by the threshold value comparisons described by means of FIG. 2, so that detection of an apnea, and in particular even the detection of the beginning of an apnea, may take place in real time using extremely simple means.

As the above-described medicinal mechanisms reveal, a pulse wave signal dependent on the blood pressure is provided in some embodiments of the invention. The pulse wave signal is evaluated so as to infer the existence of an apnea when a change in a waveform of the pulse wave signal meets a predetermined detection criterion.

In further embodiments of the present invention, sensors may be employed which have a different manner of providing a pulse wave signal which is dependent on the blood pressure. These may include, for example, highly sensitive blood pressure measurement devices and/or integrated pressure sensors. In addition, the information provided by an EEG or an ECG may be used, for example, since said information also includes information about the progress of the blood pressure. In further embodiments of the present invention, one may also evaluate a higher-frequency signal portion of the pulse wave signal so as to infer the existence of an apnea.

Such an approach may be motivated, for example, by the fact that when there is an oxygen debt, the elasticity of the arteries in the human body is reduced. Consequently, a pressure increase as is caused by the heartbeat is attenuated to a lesser degree by elastic reactions of the artery system. Consequently, a higher-frequency signal portion of the blood pressure-dependent signal (the spikes of the jagged curve 20 in FIG. 1) will also change. This is due to the fact that the pressure increase occurs considerably faster, i.e. the slope of the spikes, or the slope of the signal edges of a signal portion, describing a pulse beat, of a blood pressure-dependent pulse wave signal will increase as compared to an elastic vascular system. Consequently, alternative embodiments may also evaluate, on the basis of said information, a change in a higher-frequency signal portion of the pulse wave signal so as to infer the existence of an apnea from a change in the speed of the higher-frequency signal portion.

To this effect, FIG. 2 schematically shows a curve shape of a low-frequency portion of a pulse wave signal 30, the waveform being plotted in random units of the intensity versus the time in random units.

FIG. 2 further shows a moment of evaluation 32 (t_{A}) as well as an interval starting time 34a and an interval end time 34b, which define a time interval 36 which comes before the evaluation time 32. Detection of an apnea and, in particular, also the beginning of an apnea at the evaluation time 32 may now take place by defining, for example, an absolute threshold-value criterion, i.e. by inferring the existence of an apnea or the beginning of an apnea when a fixed threshold value 38 of the low-frequency portion of the pulse wave signal is fallen short of.

In alternative embodiments of the present invention, which additionally take into account that the low-frequency signal portion may also change as a result of other factors, a time-variable threshold value 38 is formed in that the signal within the time interval 36 which comes before the evaluation time 32 is used for calculating the threshold value 38 below which the existence of an apnea shall be inferred. This may occur, for example, by taking the mean of the pulse wave signal within the interval 36, whereupon the threshold value 38 is specified, for example, as a fixed fraction of the mean taken within the interval 36. As is shown in FIG. 2, on the one hand, the time interval 36 may come before the evaluation time 38 such that the time interval 36 does not last until the evaluation time 32. Alternatively, it is also possible, of course, to select a time interval which ends with the evaluation time 32. Alternative embodiments may also use, e.g., the temporal derivation of the low-frequency portion of the pulse wave signal as a criterion, so that the existence of an apnea may be inferred when the temporal derivation falls short of a specific value.

Alternatively, it is also possible, for example, to perform a linear regression within the time interval 36, or to adjust a polynomial of a higher order to the waveform so as to determine a detection criterion at the evaluation time 32 on the basis of the adjusted function which is extrapolated at the evaluation time 32, said detection criterion meaning that an apnea will be inferred when said detection criterion is met.

What is advantageous in all of the above-described evaluation or analysis methods is that due to the photoplethysmographically obtained pulse wave signal, the evaluation steps, or the process steps that may be used for signal processing, may be performed without any major computing expenditure, so that the apneas may be detected, on the one hand, using low-cost hardware, and, on the other hand, in real time.

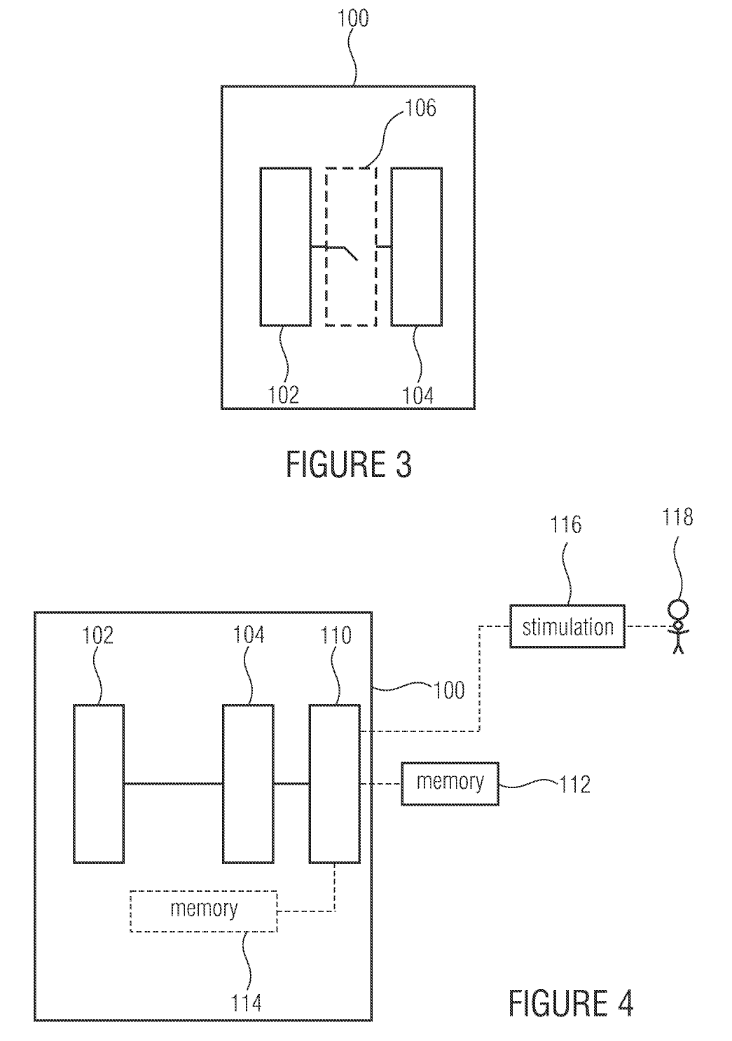

FIG. 3 shows an example of an apparatus for detecting apneas 100, which comprises a provision means 102 for providing a photoplethysmographically detected pulse wave signal, and an evaluation means 104 for evaluating the signal. The evaluation means 104 infers the existence or the beginning of an apnea when a change in a low-frequency portion of the pulse wave signal provided by the provision means 102 meets a predetermined detection criterion. Examples of such detection criteria have been discussed with reference to the previous figures.

The provision means 102 may receive, for example, the pulse wave signal that has been captured by a photoplethysmography sensor in real time, so as to provide same. Alternatively, it is possible that the provision means obtains signals that have already been recorded from an external medium or reads same from a memory so as to provide same. FIG. 3 additionally shows an optional low-pass filter 106 so as to generate the low-frequency portion of the pulse wave signal from that pulse wave signal that is provided by the provision means 106.

It is advantageous to select the crossover frequency of the low-pass filter such that it is below the pulse frequency of a human being. The crossover frequency may therefore be selected to be below 1 Hz, below 0.5 Hz or below 0.2 Hz.

In a further implementation of the invention, the crossover frequency is adaptively adjusted, since the evaluation means 104 is further configured to evaluate the pulse wave signal in terms of the pulse frequency so as to adaptively control—while knowing the current pulse frequency—the low-pass filter such that low-pass filtering is not impaired by the pulse frequency because it can be ensured that the cutoff frequency of the low-pass filter is below the pulse frequency.

In further implementations, the low-pass filter 106 may also be replaced by taking the mean within a time interval or by any other measure capable of extracting the low-frequency signal portion of the pulse wave signal provided by the provision means 102.

FIG. 4 shows a further embodiment of the present invention in an apparatus for detecting apneas 100, which also comprises, just like the apparatus shown in FIG. 3, a provision means 102 and an evaluation means 104, so that another description of the two means shall be dispensed with below. In particular, in the entire context of the present application, any components which are identical or similar in function are provided with the same reference numerals, their descriptions by means of the individual embodiments being mutually interchangeable or being transferable between individual embodiments.

The apparatus for detecting apneas 100 further comprises an alarm means 110 configured to perform and alarm action when the evaluation means has inferred the existence of an apnea or the beginning of an apnea.

FIG. 4 depicts several alternatives for an alarm action, which may also be taken at the same time, of course. One example of an alarm action comprises, for example, storing the time of the apnea detected in an external memory 112 or an internal memory 114, which may be located within the apparatus for detecting apneas.

At the same time, it is possible in an advantageous manner to store a measure of the intensity of the apnea, which measure may be determined on the basis of the detection criterion. If, for example, a threshold-value comparison is employed as the detection criterion, the magnitude of the deviation from the threshold value may be used as a measure of the severity of the apnea detected.

Since, as was already mentioned, it is also possible to detect the beginning of an apnea on the basis of the photoplethysmographic signals, further embodiments of the present invention comprise generating, as the alarm action, a stimulation signal which may cause an external stimulation means 116 to control a CPAP device in a suitable manner, so that the patient will not enter into too high an oxygen debt.

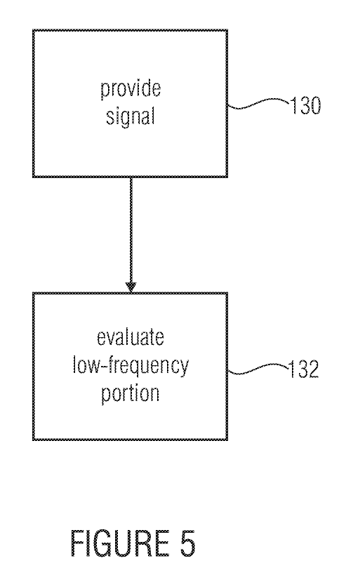

FIG. 5 shows an example of a method of detecting apneas, wherein photoplethysmographically detected pulse wave signals are provided in a provision step 130. Said signals are evaluated in a an analysis step or in an evaluation step 132 so as to infer the existence of a apnea when a change in a low-frequency portion of the pulse wave signal meets a predetermined detection criterion.

In summary, it is therefore possible, with the present invention, to detect the sleep-related breathing disturbances (the so-called sleep apneas) from a photoplethysmographically detected pulse wave or signal.

In practical operation, it is particularly advantageous that in a polysomnographic sleep study, the oxygen saturation is normally routinely detected by means of a photoplethysmographic sensor. Many other sensor sets, which record only a subset of the entire polysomnography data set include SpO₂ measurement. In clinical everyday life, therefore, apneas may now be reliably detected without the necessity to attach or buy an additional sensor. Moreover, by applying the inventive concept, a multitude of other sensors which have been used in polysomnography to date may become superfluous, or said sensors may be used for redundancy formation.

Depending on the circumstances, the inventive method may be implemented in hardware or in software. Implementation may occur on a digital storage medium, in particular a disk or CD with electronically readable control signals which may cooperate with a programmable computer system such that the inventive method is performed. Therefore, the invention generally also consists in a computer program product having a program code, stored on a machine-readable carrier, for performing the inventive method, when the computer program product runs on a computer. In other words, the invention may therefore be realized as a computer program having a program code for performing the method, when the computer program runs on a computer.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1-21. (canceled)

22. An apparatus for detecting apneas, comprising:
a provider for providing a blood pressure-dependent pulse wave signal; and
an evaluator for evaluating the pulse wave signal provided so as to infer the existence of an apnea when a low-frequency portion of the pulse wave signal falls below a predetermined threshold value,
the evaluator being configured to determine the time-variable threshold value for an evaluation time by averaging the low-frequency portion of the pulse wave signal within a time interval which comes before the evaluation time.

23. The apparatus as claimed in claim 22, wherein the provider is configured to provide a photoplethysmographically detected pulse wave signal.

24. The apparatus as claimed in claim 22, further comprising:
a low-pass filter for deriving the low-frequency portion of the pulse wave signal from the pulse wave signal.

25. The apparatus as claimed in claim 24, wherein the low-pass filter comprises a cutoff frequency which is below a pulse frequency.

26. The apparatus as claimed in claim 24, wherein the evaluator is configured to evaluate the pulse wave signal in terms of the pulse frequency so as to control the cutoff frequency of the low-pass filter such that said cutoff frequency is below the pulse frequency.

27. The apparatus as claimed in claim 22, wherein the provider comprises a photoplethysmography sensor.

28. The apparatus as claimed in claim 27, wherein the photoplethysmography sensor is configured to employ light of a single wavelength range.

29. The apparatus as claimed in claim 22, wherein the provider comprises a memory interface for reading out a pulse wave signal stored on an external medium.

30. The apparatus as claimed in claim 22, wherein the evaluator comprises a differentiator so as to determine a temporal derivation of the low-frequency portion of the pulse wave signal, the evaluator being configured to infer the existence of an apnea when the temporal derivation falls below the predetermined threshold value.

31. The apparatus as claimed in claim 22, comprising an alarm so as to perform an alarm action when the evaluator has inferred the existence of an apnea.

32. The apparatus as claimed in claim 31, wherein the alarm is configured to store, as an alarm action, the time of the detection of an apnea in an external memory.

33. The apparatus as claimed in claim 31, wherein the alarm is configured to store, as an alarm action, a measure of the severity of the detected apnea on the basis of the detection criterion.

34. The apparatus as claimed in claim 31, wherein the alarm is configured to activate, as an alarm action, a ventilation support, or to increase the support function thereof.

35. A method of detecting apneas, comprising:
providing a blood pressure-dependent pulse wave signal; and
evaluating the pulse wave signal provided so as to infer the existence of an apnea when a low-frequency portion of the pulse wave signal falls below a predetermined threshold value, the time-variable threshold value for an evaluation time being determined by averaging the low-frequency portion of the pulse wave signal within a time interval which comes before the evaluation time.

36. A computer readable medium having a computer program with program code for executing, when the computer program runs on a computer, a method of detecting apneas, said method comprising:
providing a blood pressure-dependent pulse wave signal; and
evaluating the pulse wave signal provided so as to infer the existence of an apnea when a low-frequency portion of the pulse wave signal falls below a predetermined threshold value, the time-variable threshold value for an evaluation time being determined by averaging the low-frequency portion of the pulse wave signal within a time interval which comes before the evaluation time.
